(19) United States
(12) Patent Application Publication
(10) Pub. No.: US 2025/0352242 A1
(43) Pub. Date: Nov. 20, 2025

(54) INSERTION SYSTEM AND METHOD FOR INSERTING A MEDICAL DEVICE

(71) Applicant: Roche Diabetes Care, Inc., Indianapolis, IN (US)

(72) Inventor: Thomas Kuensting, Mannheim (DE)

(21) Appl. No.: 19/285,164

(22) Filed: Jul. 30, 2025

Related U.S. Application Data
(63) Continuation of application No. PCT/EP2024/053557, filed on Feb. 13, 2024.

(30) Foreign Application Priority Data
Feb. 15, 2023 (EP) 23156733.0

(51) Int. Cl. A61B 17/34; A61B 5/1473; A61B 17/00; A61L 31/12; A61L 31/14; A61M 5/158; A61M 27/00

(52) U.S. Cl. CPC A61B 17/3468; A61B 5/1473; A61L 31/125; A61L 31/14; A61M 5/158; A61M 27/006; A61B 2017/00526; A61B 2560/063; A61M 2005/1585; A61M 2027/004

(57) ABSTRACT

Insertion tool for inserting at least a part of a medical device into a subject, insertion device comprising the insertion tool and an insertion mechanism, and method for manufacturing the insertion tool, wherein the insertion tool comprises a penetrating portion that comprises a composite material comprising at least one first material and at least one second material. Wherein the first material is an amorphous material or amorphous composite and the second material is a fibrillary material or fibrillary composite or a crystalline material or crystalline composite. A fluid absorption of the first material of the composite material is higher than a fluid absorption of the second material of the composite material, and the insertion device is adapted to soften when in contact with a body fluid at least due to the fluid absorption of the amorphous material or amorphous composite.

### CROSS REFERENCE TO RELATED APPLICATIONS

This is a continuation of PCT/EP2024/053557 filed Feb. 13, 2024, which claims priority from EP 23 156 733.0 filed Feb. 15, 2023, the disclosures of both of which are hereby incorporated herein by reference.

### BACKGROUND

The present application refers to an insertion tool for inserting at least a part of medical device into a subject.

For the proper management of chronic health conditions, it may be crucial for a subject to periodically monitor one or more analyte levels in his or her blood stream and/or his or her interstitial fluid. In the case of diabetes, the subject, i.e., patient, routinely monitors the glucose levels to avoid hypoglycemic episodes as well as hyperglycemic episodes. For other situations where health monitoring is important, other analytes, such as lactate, cholesterol, oxygen or other types of analytes and metabolites, may be measured.

Monitoring systems have been developed which allow a sensor to be implanted into a subject to monitor the analyte concentration directly within the bloodstream or within the interstitial fluids. An assembly for inserting such a sensor is known from WO 2011/041449 A1. The insertion assembly comprises a rigid introducer sharp as an insertion tool for piercing the subject's skin and positioning the sensor within the subject's connective tissue. After the insertion of the sensor, the insertion tool is removed by a retraction mechanism of the insertion assembly.

Other medical devices to be partly or totally implanted into a subject are known.

US 2012/0276221 A1 discloses an insertable medical device wherein at least a part of the medical device softens or completely dissolves upon implantation or insertion into a subject. As an embodiment, a stent is described with a tapered tip, to aid insertion, whereas the stent is at least partially formed from a fully urine-disintegrable polymer or a biodisintegrable polymer blended with a biostable polymer. A multitude of examples for biostable are biopolymers, such as polypeptides, proteins, and polysaccharides, including fibrin, fibrinogen, collagen, elastin, chitosan, gelatin, starch, glycosaminoglycans such as hyaluronic acid.

Another example for a medical device changing its properties after insertion is given in US 2018/0207356 A1 disclosing a subcutaneous infusion catheter. The catheter comprises a flexible cannula with an outer wall changing from smooth to an accordion-like shape thereby moving or pulling the needle tip back from its end position, i.e., from the area of most trauma, and helping to prevent the cannula from pulling out of the skin as well as providing more surface area for delivery of insulin.

U.S. Pat. No. 7,513,891 B2 describes a cannula comprising a temperature-sensitive medium that causes the cannula to be rigid prior to insertion and to be flexible after insertion due to body temperature. Either the whole cannula with tip is formed of the temperature-sensitive material or the temperature-sensitive medium may be received in a porous material forming the cannula or within a cleared space between different soft tube-like parts of the cannula. Examples for suitable materials are PTFE, PUR or SR.

Another example of an injection needle changing from a rigid pre-insertion state to a flexible state after insertion is known from U.S. Pat. No. 7,435,240, B2. The transition can, in particular, be based on the change in temperature or pH or on a chemical reaction of the material with a surrounding medium, or on a combination of several of these factors. Materials named are thermoplastic polymers or polymers with a glass transition.

Another example of a needle dissolving after insertion may be found in U.S. Pat. No. 9,675,545 B2 the needle comprising a chitosan derivative, i.e., a biopolymer, selected from chitosan succinamide, carboxymethyl chitosan, trimethyl chitosan, and a combination thereof.

It is therefore desirable to provide an insertion tool which at least partially addresses the above-mentioned technical challenges and provide an advantageous alternative to the known solutions.

### SUMMARY

This problem is addressed by an insertion tool described herein. Advantageous embodiments which might be realized in an isolated fashion or in any arbitrary combinations are also described throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, an insertion tool for inserting at least a part of a medical device into a subject is disclosed, wherein at least a penetrating portion of the insertion tool softens when in contact with a body fluid at least due to fluid absorption.

“Medical device” refers to an arbitrary element or article being configured for use in the field of medical technology, specifically in the field of medical analytics or medical diagnostics. The medical device may be configured for performing at least one medical function and/or for being used in at least one medical process, such as one or more of a therapeutic process, a diagnostic process or another medical process.

For example, the medical device may comprise at least one of: a device for delivery of at least one therapeutic fluid, for example insulin, into the body tissue of the user, specifically at least one infusion set comprising at least one infusion cannula; at least one analyte sensor for detecting at least one analyte, for example blood glucose or a pH value; specifically at least one analyte sensor for detecting at least one bodily analyte in the at least one bodily fluid, more specifically at least one electrochemical analyte sensor comprising at least one sensor electrode for detecting the at least one analyte.

“Insertion tool” refers to a member suitable to enter a subject's skin for being inserted. “Insertion tool” may, for example, refer to a needle-like or tube-like structure having a sharp or solid tip or the like when being inserted. The insertion tool or at least a part of the insertion tool may be part of the medical device, e.g., the insertion tool may be a cannula or a distal part of a cannula for drug administration. In another embodiment the insertion tool may form a substrate or bases for the medical device or a part of the medical device or the insertion tool may receive the medical device or a part of the medical device for insertion of the medical device. The insertion tool may insert the whole medical device or a portion or part of the medical device. Inserting a portion of the medical device may comprise positioning a non-insertable part of the medical device onto the skin of the subject.

“Penetrating portion” refers to a tip portion of the insertion tool, i.e., the distal part of the insertion tool adapted to enter a subject's skin during an insertion process.

The insertion tool may be inserted manually or using an insertion device, wherein “Insertion device” refers to a device configured for inserting the insertion tool and therewith the medical device fully or partially into the body tissue, i.e., comprising an insertion mechanism to insert the insertion tool. The insertion device may be configured for transcutaneous or subcutaneous insertion of the insertion tool, such as by pushing the penetrating portion of the insertion tool into the body tissue of the user by performing an incision or a puncture in the skin.

“Fluid absorption” refers to a physical and/or chemical or reactive phenomenon or process in which a fluid, i.e., the fluids molecules, atoms and ions, is taken up or retained by another material, i.e., enters the bulk phase of the other material, e.g., a solid material.

In one aspect of the disclosure, the penetrating portion comprises a composite material that includes at least one first material and at least one second material, wherein the first material is an amorphous material or amorphous composite, the second material is a fibrillary material or fibrillary composite or a crystalline material or crystalline composite, and wherein a fluid absorption of the first material of the composite material is higher than a fluid absorption of the second material of the composite material. In other words, the composite material comprises a first portion consisting of one or more amorphous material or amorphous composites and a second portion consisting of one or more fibrillary material or fibrillary composites and/or crystalline material or crystalline composites. By combining the fibrillary or crystalline material or crystalline composite or a group of fibrillary and/or crystalline material or crystalline composites with an amorphous material or amorphous composite or a group of amorphous material or amorphous composites a composite is created that shows a high rigidity in ambient, i.e., dry, conditions due to its portion of fibrillary and/or crystalline material(s) and softens when exposed to a fluid due to fluid absorption which is enhanced due to its portion of amorphous material or amorphous composite(s) or materials.

“Composite material” or “composition material” or “composite” refers to a material which comprises at least a first material and a second material as constituent materials, wherein the two or more constituent materials have notably dissimilar chemical or physical properties and are merged to form a finished structure in which they remain separate and distinct. In other words, the starting materials are connected to each other but a solution of the individual starting materials with one another does not take place or only takes place superficially. For example, particles or fibers of a first component are embedded in another component of the composite material, forming a matrix structure. In fiber composite materials, the fibers can extend in one or more specific directions or have preferred directions. Fiber composite material can also be produced in layers.

The penetrating portion comprises the composite material, in particular the penetrating portion fully or substantially consists of the composite material, wherein here “substantially” refers to the overall portion of composite material within the penetrating portion dominating the behavior of the penetrating portion with respect to fluid absorption and softening.

The constituent materials of the composite material of the insertion tool's penetrating portion are an amorphous material or amorphous composite and a fibrillary or crystalline material or composite. This does not exclude that the composite material contains other materials as long as the amount of the other materials do not substantially change the composite material's behavior with respect to fluid absorption, rigidity and softening.

“Amorphous material or amorphous composite” or “non-crystalline material or composite”, refers to a solid that lacks the long-range order, which is a characteristic of a crystal or a crystalline material or crystalline composite. The amorphous material or amorphous composite, i.e., a composite of at least two amorphous materials, has an internal structure comprising interconnected structural blocks, wherein the structural blocks may, e.g., be similar to the basic structural units found in a corresponding crystalline phase of the same compound. Whether a material is liquid or solid depends primarily on the connectivity between its elementary building blocks; solids are characterized by a high degree of connectivity whereas structural blocks in fluids have lower connectivity. Here the amorphous material or amorphous composite comprised by the composite material is in a solid state.

“Crystalline material or crystalline composite” or “crystal” refers to a solid material or a composite of solid materials whose constituents such as atoms, molecules, or ions are arranged in a highly ordered microscopic structure, forming a crystal lattice that extends in all directions. “Fibrillary material or fibrillary composite” or “fibrils” refers to structural biological material or a composite of structural biological materials composed of linear biopolymers, and is characterized by rod-like structures with a high length-to-diameter ratio.

While the crystalline or fibrillary material or fibrillary composite portion is characterized by a certain hardness giving the insertion tool stability, the amorphous material or amorphous composite portion allows the absorption of a greater amount liquid or fluid, in particular of a body fluid, and thus the softening of the insertion tool. Thus, in normal ambient conditions, i.e., average humidity, temperature, pressure etc., the penetrating portion of the insertion tool is in a rigid or stiff state and therewith adapted to puncture or prick a subject's skin for insertion of the medical device. After being inserted, the penetrating portion softens due to the absorption of the surrounding body fluid. Therewith, the insertion tool may remain within the subject's skin for the whole time the medical device is left on and/or in the subject's skin without causing any discomfort or pain. A retraction of the insertion tool while dissembling from the medical device and leaving the medical device inserted is not necessary, thus, significantly simplifying the insertion process and the requirements for an insertion device, if used.

The fluid absorption of a material, i.e., the ability to absorb a certain amount of fluid, depends for example on the inner structure of the material. For example, the water absorption of a material and thus its softening properties may depend primarily on an amount of cross-linking monomeric components within the material, such as disulfides, also called disulfide bridges or ss-bonds and does not depend on a degradation of the respective material. Furthermore, the softening properties due to fluid absorption are reversible upon drying, e.g., after retraction of the insertion tool.

In an embodiment, a fluid absorption of the first material of the composite material is at least 70% of its weight in a dry state and the fluid absorption of the second material of the composite material is at most 35% of its weight in the dry state.

“Dry state” refers a first state of the first and second material when it is surrounded by air or another gas with an at least non-condensing relative humidity and/or a humidity falling below a threshold. For example, the dry state may be reached for ambient dry conditions, i.e., for air with an average humidity, temperature, pressure, etc. and not directly in contact with a fluid or tissue or the like. In another embodiment, the dry state may be reached for air with a lower humidity, e.g., with a humidity reduced and or controlled by desiccants or the like that are added to a packaging of the insertion tool. The dry state of the first and second material, respectively, may be defined by an overall water content of the material such as, for example, 10 wt % water overall, i.e., in the dry state a mass fraction of 10% of the overall or total mass of the material is water.

In an embodiment, the amorphous material or amorphous composite portion is adapted to absorb, i.e., to take up, at least an amount of water corresponding to 70% of its weight in the dry state, i.e., of the amorphous material or amorphous composite portion's weight in the dry state. Correspondingly the crystalline or fibrillary material or fibrillary composite portion is adapted to absorb at most an amount of water corresponding to 35% of its weight in the dry state, i.e., of the crystalline or fibrillary material or fibrillary composite portion's weight in the dry state.

In another embodiment, the fluid absorption of the first material of the composite material is at least 60% or at least 65% or at least 75% or at least 80% of its weight in a dry state. The fluid absorption of the amorphous material or amorphous composite portion of the composite material allows for the softening of the penetrating portion.

In a further embodiment, the fluid absorption of the second material of the composite material is at most 40% or at most 30% or at most 25% of its weight in the dry state. The second material portion of the composite material allows for the penetrating portion's rigidity in ambient conditions to enable the penetration of a subject's skin. A low ability to

In an embodiment, a weight percent (wt %) of first material and second material with regard to the composite material in a dry state are equal or 20/80 or 80/20, i.e., the portion of the weight of the composite material in the dry state of the first material is at least 20% and at most 80%, while the portion of the weight of the composite material in the dry state of the second material is at most 80% and at least 20%.

In another embodiment, the first material and the second material are homogeneously distributed within the composite material or are at least in parts structured.

In an embodiment, a distribution of the second material within the composite material of the insertion tool is oriented along a longitudinal axis of the insertion tool.

The longitudinal axis typically points in the direction of insertion. With the second crystalline or fibrillary material or fibrillary composite proceeding along the longitudinal axis, i.e., the direction of insertion, the penetrating portion's stability in the direction of insertion may be increased.

In another embodiment, the first material and/or the second material is a biopolymer. “Biopolymer” refers to natural polymers produced by the cells of living organisms. For example, fibrin or keratin. A biopolymer may help to increase the biocompatibility of the composite material, i.e., of the penetrating portion, wherein here biocompatibility refers to the ability of a material to perform with an appropriate host response, e.g., to reduce an immune response in the subject when implanted. In other words, the biocompatibility refers to the quality of not having toxic or injurious effects on biological systems.

In a further embodiment, the first material and/or second material is keratin. Keratin is an example of a biopolymer and has the properties of biocompatibility, biodegradability, bioactivity and it possesses a hydrophilic surface, which is absent in many synthetic polymers and enhances its capacity to absorb a fluid. Keratin is a fibrous protein that for example is a key structural material of hair, nails, horns, claws, and hooves.

In another embodiment, the first material is keratin and the first material in a dry state contains at least 10 wt % cysteine. “Cysteine” refers to a sulfur-containing proteinogenic amino acid with the formula HOOC—CH(—NH₂)—CH₂—SH and is typically present in keratin in large amounts forming so called disulfide bridges and therewith conferring additional strength and rigidity by permanent, thermally stable crosslinking. “Disulfide bridge” refers to a covalent link between the sulphur atoms of two cysteine amino acids and their formation stabilizes the tertiary and higher order structure of proteins. Disulfides in proteins are formed between the thiol groups of cysteine residues by the process of oxidative folding. The prototype of a protein disulfide bond is the two-amino-acid peptide cystine, which is composed of two cysteine amino acids joined by a disulfide bond. The structure of a disulfide bond can be described by its χₛₛ dihedral angle between the C^{β}—S^{γ}—S^{γ}—C^{β} atoms, which is usually close to ±90°. In another embodiment the first material contains in the dry state at least 15 wt % cysteine or at least 20 wt % cysteine.

In a further embodiment, the first material contains a first amount of a first cross-linking monomeric component or of different types of cross-linking monomeric components, and the second material comprises a second amount of a second cross-linking monomeric component or of different types of cross-linking monomeric components, wherein the first amount is smaller than the second amount. Each amount may be between to 0 wt % or 100 wt % with regard to dry state. The first and second cross-linking monomeric component may be identical, e.g., disulfide bridges. Via the amount of cross-linking monomeric components the water absorption capacity and therewith the softening properties may be controlled, e.g., chosen beforehand when manufacturing the penetrating portion of the medical device.

In an embodiment, the first cross-linking monomeric component and/or the second cross-linking monomeric component is cysteine.

In another embodiment, the first material comprises a first amount of disulfide, the second material comprises a second amount of disulfide, wherein the first amount is smaller than the second amount. The amount of disulfide bridges may, for example, be controlled by the amount of a cross-linking monomeric component, e.g., cysteine, as the cross-linking monomeric components, like cysteine, are required to form the disulfide bridges.

In an embodiment, the penetrating portion is adapted to have a rigid state and a soft state, in the rigid state an overall fluid content of the composite material of the penetrating portion is at most 10 wt % or at most 20 wt % and in the soft state an overall fluid content of the composite material of the penetrating portion is at least 50 wt % or at least 60 wt %.

In another aspect, an insertion device comprising an insertion tool as described above, and an insertion mechanism for penetrating the insertion tool into the subject and therewith inserting the medical device or the portion of the medical device into the subject.

“Insertion device” refers to a device configured for inserting the insertion tool and therewith the medical device fully or partially into the body tissue, i.e., comprising an insertion mechanism to insert the insertion tool. The insertion device may be configured for transcutaneous or subcutaneous insertion of the insertion tool, such as by pushing the penetrating portion of the insertion tool into the body tissue of the user by performing an incision or a puncture in the skin.

In another aspect, a medical device comprises an insertion tool as described above.

In a further aspect, a method for manufacturing an insertion tool as described above, comprises the steps of preparing the first material with a first amount of one or more cross-linking monomeric components and preparing the second material with a second amount of one or more cross-linking monomeric components. One or more of the cross-linking mono-meric components may, for example, be cysteine. The composite material is, for example, manufactured by an additive manufacturing process such as three-dimensional printing, e.g., extrusion.

Other technical features may be readily apparent to one skilled in the art from the following figures, descriptions, and claims.

### BRIEF DESCRIPTION OF THE DRAWINGS

To easily identify the discussion of any particular element or act, the most significant digit or digits in a reference number refer to the figure number in which that element is first introduced.

FIG. 1 illustrates a composite material in accordance with one embodiment.

FIG. 2 illustrates a medical device with an insertion tool in accordance with another embodiment.

FIG. 3 illustrates a part of a penetrating portion in accordance with a further embodiment.

FIG. 4 illustrates another medical device with an insertion tool in accordance with a further embodiment.

FIG. 5 illustrates a further medical device with an insertion tool in accordance with another embodiment.

Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

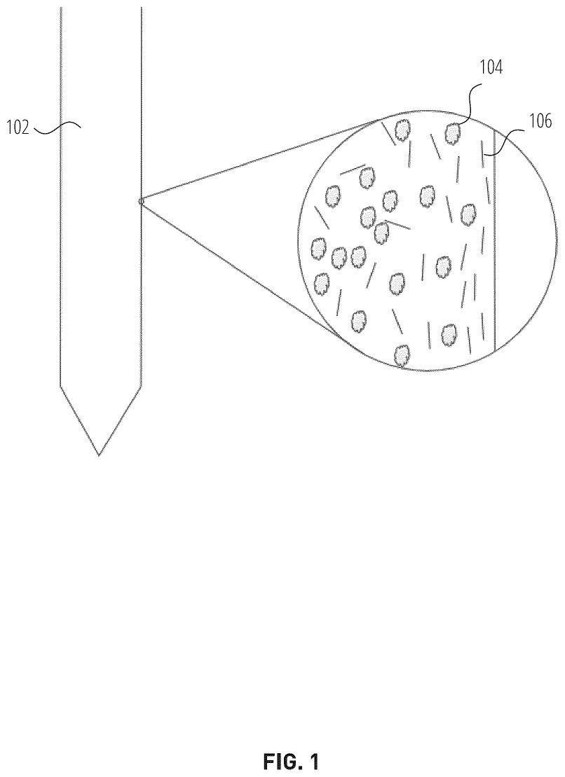

FIG. 1 schematically shows a penetrating portion 102 of an insertion tool for inserting at least a part of a medical device into a subject. In the depicted embodiment, the penetrating portion 102 is formed as a tip portion of a needle like structure and substantially consists of a composite material that comprises a first material and a second material. The first material is an amorphous material or amorphous composite 104 having a first fluid absorption ability. The second material is a fibrillary or crystalline material or crystalline composite 106 and has a second fluid absorption ability, wherein the first fluid absorption ability is greater than the second, i.e., the fluid absorption of the first material of the composite material is higher than a fluid absorption of the second material of the composite material. Due to the fluid absorption the insertion device is adapted to soften when in contact with a body fluid.

As schematically depicted in the FIG. 1 the first and second material do not form bonds but remain separate and distinct within the composite material, so that the first and second material each keep their notably different chemical and/or physical properties. While in a dry state, e.g., surrounded by air with an average humidity, temperature, pressure, etc., the fibrillary or crystalline material or crystalline composite 106 portion of the penetrating portion 102 ensure a rigidity of the penetrating portion 102 sufficient to pierce into a subject's skin, while the amorphous material or amorphous composite 104 portion of the penetrating portion 102 ensures an amount fluid absorption to soften the structure of penetrating portion 102 when surrounded by a fluid such as interstitial fluid and/or the subject's tissue, e.g., subcutaneous tissue.

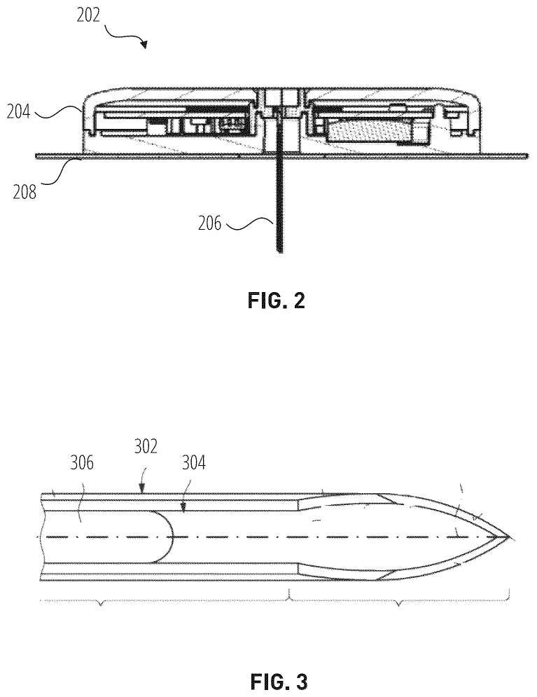

FIG. 2 shows a medical device with an interstitial analyte sensor, e.g., a glucose sensor assembly, with an upper non-insertable portion 204 and a lower penetrating portions 206. While the penetrating portion 206 is inserted into a subject's skin, the upper non-insertable portion 204 is fixed to the subject's skin via a plaster 208. The penetrating portion 206 comprises the composite material comprising of an amorphous material or amorphous composite portion and a crystalline or fibrillary material or fibrillary composite portion and an insertable part of the analyte sensor, e.g., two or three electrodes. The electrodes may be printed on a surface of the composite material or on another material covering the composite material or the like. In the dry state the penetrating portion 206 is rigid and capable of pricking a subject's skin, therewith enabling the insertion of the penetrating portion 206 either manually or using a tool such as an inserter comprising an insertion mechanism. After insertion, i.e., in contact with body tissue and bodily fluid, the composite material of the penetrating portion 206 softens due to absorption of body fluid, e.g., interstitial fluid, and remains with the insertable part of the analyte sensor in the subject's tissue.

FIG. 3 shows a distal part of a penetrating portion 302 of an insertion tool according to another embodiment. The penetrating portion 302 is formed as a hollow needle with a longitudinal cut 304, e.g., for insertion of a drug 306 that is positioned within the hollow needle and may slowly dissolve through the cut. At least the distal part of the penetrating portion 302 comprises the composite material so that the distal portion is rigid in the dry state enabling insertion into a subject's skin and softens after insertion due to absorption of body fluid. Such a hollow or at least partly hollow insertion tool may also be used to insert a sensor or the like by positioning the sensor within the void in penetrating portion 302 of the insertion tool.

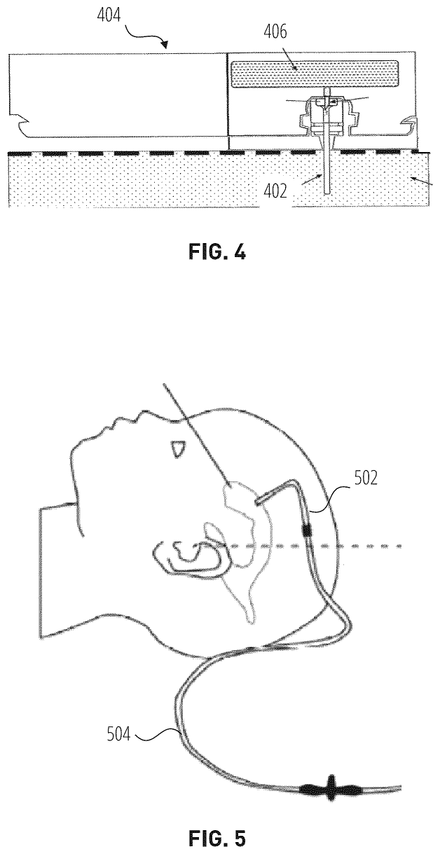

FIG. 4 shows a medical device in accordance with a further embodiment. The medical device comprises a penetrating portion formed as a cannula 402 for subcutaneous drug delivery, e.g., for insulin delivery, that consist of the composite material and therefore, is soft due to fluid absorption in the depicted inserted state. Before the insertion in a dry state, e.g., surrounded by ambient conditions and/or surrounded by air with a controlled reduced humidity, the cannula 402 is rigid and therefore may be inserted without the help of an additional insertion tool. The medical device further comprises a not-insertable upper part 404 with a drug reservoir 406, which is in fluid connection with the cannula 402.

FIG. 5 shows a medical device in accordance with a further embodiment. The medical device comprises an external ventricular drainage catheter 504 with a tubular penetrating portion 502 with a tip adapted to be placed in a cerebral ventricle to relieve elevated intracranial pressure by diverting fluid from the ventricle via an outer upper part 404 of the catheter. The penetrating portion 502 comprises the composite material so that the penetrating portion 502 and in particular its tip are rigid in a dry state, e.g. in ambient conditions, to facilitate the insertion into the brain. The penetrating portion 502 and its tip then soften due to fluid absorption.

While this invention has been described as having an exemplary design, the present invention may be further modified within the spirit and scope of this disclosure. This application is therefore intended to cover any variations, uses, or adaptations of the invention using its general principles.

What is claimed is:

1. An insertion tool for inserting at least a part of a medical device into a subject, wherein the insertion tool comprises:
a penetrating portion comprising a composite material, the composite material comprising a first material and a second material, wherein:
the first material is an amorphous material or amorphous composite,
the second material is a fibrillary material or fibrillary composite or a crystalline material or crystalline composite,
a fluid absorption of the first material of the composite material is higher than a fluid absorption of the second material of the composite material, and
the insertion device is adapted to soften when in contact with a body fluid at least due to the fluid absorption of the amorphous material or amorphous composite.

2. The insertion tool of claim 1, wherein a fluid absorption of the first material of the composite material is at least 70% of its weight in a dry state and the fluid absorption of the second material of the composite material is at most 35% of its weight in the dry state.

3. The insertion tool of claim 1, wherein a weight percent (wt %) of first material and second material with regard to the composite material in a dry state are equal or 20/80 or 80/20.

4. The insertion tool of claim 1, wherein the first material and the second material are homogeneously distributed within the composite material or are at least in parts structured.

5. The insertion tool of claim 1, wherein a distribution of the second material within the composite material of the insertion tool is oriented along a longitudinal axis of the insertion tool.

6. The insertion tool of claim 1, wherein the first material and/or the second material is a biopolymer.

7. The insertion tool of claim 1, wherein the first material and/or second material is keratin.

8. The insertion tool of claim 1, wherein the first material is keratin and the first material in a dry state contains at least 10 wt % cysteine.

9. The insertion tool of claim 1, wherein the first material contains a first amount of a first cross-linking monomeric component or of different types of cross-linking monomeric components, and the second material comprises a second amount of a second cross-linking monomeric component or of different types of cross-linking monomeric components, wherein the first amount is smaller than the second amount.

10. The insertion tool of claim 9, wherein the first cross-linking monomeric component and/or the second cross-linking monomeric component is cysteine.

11. The insertion tool of claim 1, wherein the first material comprises a first amount of disulfide, the second material comprises a second amount of disulfide and the first amount is smaller than the second amount.

12. The insertion tool of claim 1, wherein the penetrating portion is adapted to have a rigid state and a soft state, wherein in the rigid state an overall fluid content of the composite material of the penetrating portion is at most 20 wt % and in the soft state an overall fluid content of the composite material of the penetrating portion is at least 50 wt %.

13. The insertion tool of claim 12, wherein the overall fluid content of the composite material of the penetrating portion in the rigid state is at most 10 wt %.

14. The insertion tool of claim 12, wherein the overall fluid content of the composite material of the penetrating portion in the soft state is at least 60 wt %.

15. The insertion tool of claim 14, wherein the overall fluid content of the composite material of the penetrating portion in the rigid state is at most 10 wt %.

16. An insertion device comprising:
the insertion tool of claim 1, and
an insertion mechanism for penetrating the insertion tool into the subject and therewith inserting the medical device or the portion of the medical device into the subject.

17. A medical device comprising the insertion tool of claim 1.

18. A method for manufacturing the insertion tool of claim 1, wherein the first material is prepared with a first amount of one or more cross-linking monomeric components and the second material is prepared with a second amount of one or more cross-linking monomeric components.
